(19) United States
(12) Patent Application Publication
(10) Pub. No.: US 2010/0292278 A1
(43) Pub. Date: Nov. 18, 2010

(54) Methods of treating vascular disease and injury

(76) Inventors: Yochai Birnbaum, Houston, TX (US); Yumei Ye, League City, TX (US); J.Regino Perez-Polo, Galveston, TX (US)

Correspondence Address: Benjamin Aaron Adler;ADLER & ASSOCIATES, 8011 Candle Lane, Houston, TX, 77071, US

(21) Appl. No.: 12/800,520

(22) Filed: May 17, 2010

Related U.S. Application Data
(60) Provisional application No. 61/216,356, filed on May 15, 2009.

(51) Int. Cl. A61K 31/4439; A61K 31/7088; A61P 9/10

(52) U.S. Cl. 514/342; 514/44 A

(57) ABSTRACT

The present invention provides a method of treating a vascular disease or injury in an individual in need of such treatment, comprising the step of administering a compound or composition that down-regulates or decreases the activity of microRNA-29 in the individual. Also provided is a method of down-regulating or decreasing the activity of microRNA-29 in an individual in need of such treatment, comprising the step of administering a PPAR-γ agonist compound or PPAR-γ agonist containing composition that down-regulates or decreases the activity of microRNA-29 in the individual. Further provided is a method of treating a vascular disease or injury in an individual in need of such treatment, comprising the step of administering a microRNA-29 antagonist that down-regulates or decreases the activity of microRNA-29 in said individual.

### CROSS-REFERENCE TO RELATED APPLICATIONS

This non-provisional applications claims benefit of priority under 35 U.S.C. §119(e) of provisional U.S. Ser. No. 61/216,356, filed May 15, 2009, now abandoned, the entirety of which is hereby incorporated by reference.

### BACKGROUND OF THE INVENTION

1. Field of the Invention

The present invention generally relates to the fields of cardiovascular physiology and pathology. More particularly, the present invention relates to novel methods of treating cardiovascular and cerebrovascular diseases and injuries.

2. Description of the Related Art

MicroRNAs (miRNAs) are a family of short non-coding RNAs that pair with specific “target” mRNAs and negatively regulate their expression through translational repression or mRNA degradation (1-3). Recent studies have shown that miRNAs control diverse aspects of cardiac functions, including hypertrophy, fibrosis, remodeling, heart failure and arrhythmia (2-7). Thus, miRNAs are attractive potential targets for therapy of diverse cardiac conditions. However, the role of miRNAs in modulating ischemia reperfusion injury (IRI) has not been fully determined. There is one study assessing the effect of H₂O₂ exposure on miRNA expression (8).

There is a recognized need in the art for improved therapeutic treatments against cardiovascular and cerebrovascular diseases and injuries such as ischemia-reperfusion injury. The present invention fulfills this longstanding need and desire in the art.

### SUMMARY OF THE INVENTION

In one embodiment, the present invention provides a method of treating a vascular disease or injury in an individual in need of such treatment, comprising the step of administering a compound or composition that down-regulates or decreases the activity of microRNA-29 in said individual.

In another embodiment, the present invention provides a method of down-regulating or decreasing the activity of microRNA-29 in an individual in need of such treatment, comprising the step of administering a PPAR-γ agonist compound or PPAR-γ agonist containing composition that down-regulates or decreases the activity of microRNA-29 in said individual.

In another embodiment, the present invention provides is a method of treating a vascular disease or injury in an individual in need of such treatment, comprising the step of administering a microRNA-29 antagonist that down-regulates or decreases the activity of microRNA-29 in said individual.

Other and further aspects, features, and advantages of the present invention will be apparent from the following description of the previously preferred embodiments of the invention. These embodiments are given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1F shows mRNAs expression changes after administration of 5 mg/kg per day pioglitazone (PIO). FIG. 1A shows the log 2 value of each PIO/sham pair of miRNA microarray signal were displayed in a heat-map generated by TIGR Multiexperiment Viewer software. Red indicates upregulation; green, downregulation; black, no change. The bar code on the top represents the color scale of the log 2 values. FIG. 1B is a bar graph indicates the fold change in expression in PIO group compared with the baseline of the miRNAs of interest. Northern blot confirmed that PIO time-dependently decreased miR-29a (FIGS. 1C, 1E) and miR-29c (FIGS. 1D, 1F) levels in rat hearts. FIGS. 1C-1D show samples of Northern blots of miR-29a and miR-29c, respectively. FIGS. 1E-1F are a densitometry analysis of myocardial levels of miR-29a and miR-29c, respectively. * p<0.05 versus control.

FIG. 2A-2B show Mir-29a (FIG. 2A) and MiR-29c (FIG. 2B) expression changes in H9c2 myocytes after PIO and GW9662 (a PPAR-γ antagonist) treatment as determined by qRT-PCR. Note: Mean miRNA levels in vehicle control were defined as 100%. The small housekeeping RNA U6 was used as a loading control. *P<0.05 compared with those in control. Four independent experiments were performed.

FIG. 3A-3D demonstrate that MiR-29 is involved in the protective effect of PIO against simulated ischemia reoxygenation (SIR). H9c2 cells were treated with: 1) Control (2% ethanol); 2) PIO 10 nM; 3) PIO+mim-29a; 4) PIO+mim-29c and 5) PIO+mim-29a+mim-29c. FIG. 3A shows cell viability was assessed by MTT assay. Results represent means ± S.E.M., expressed as percentage of control, taken as 100% (n=8 in each group). Overall, there were significant differences among groups, both in cells not exposed to SIR(NSIR, p<0.001) and in cells exposed to SIR (p<0.001). * p<0.05 versus control NSIR. # p<0.05 versus control SIR.\ p<0.05 versus PIO+mim 29a+mim-29c SIR. FIG. 3B shows the percentage of dead cells after exposure to SIR, assessed by trypan blue staining. Results represent means ±S.E.M. of the percent of positively stained cells (N=8 in each group). Overall, there were significant differences among groups (p<0.001). * p<0.5 versus control. # p<0.05 versus PIO. FIG. 3C is a quantitative assay of apoptotic cells (TUNEL assay) after exposure to SIR. Results represent means ± S.E.M., expressed as percentage of control, taken as 100% (n=6 in each group). Overall, there were significant differences among groups (p<0.001). * p<0.05 versus control. # p<0.05 versus PIO. FIG. 3D shows Caspases-3 activity (n=4 in each group). Overall, there were significant differences among groups (p<0.001). * p<0.05 versus control. # p<0.05 versus PIO.

FIG. 4A-4F: Effect of miR-29 on viability of H9c2 cells subjected to SIR or NSIR. FIGS. 4A-4B show that miR-29 expression levels were assessed by Northern Blot after 24 hs transfection of mimic (mim-29), inhibitor (inh-29), negative control for mimic (NC-mim) and inhibitor (NC-inh). The small housekeeping RNA U6 (106 nt) was used as a loading control. FIGS. 4C-4D show the MTT viability in cells subjected to SIR or NSIR. Results represent means ± S.E.M. (N=8 in each group), expressed as percentage of control (N SIR), taken as 100%. Overall, there were significant differences among the treatment groups in both the cells not exposed to SIR(NSIR, p<0.001) and those exposed to SIR (p<0.001). * p<0.05 versus NC-mim NSIR. # p<0.05 versus NC-mim SIR. FIGS. 4E-4F show that cell death was assessed by trypan blue staining. The results represent means ± S.E.M. of the percentage of cells stained positive (N=6 in each group). There was no difference among the treatment groups in cells not exposed to SIR(NSIR; p=0.974 for miR-29a and p=0.828 for miR-29c). Among cells subjected to SIR there were significant differences among groups (p<0.001 for miR-29a and p<0.001 for miR-29c). * p<0.05 versus control SIR.

FIG. 5A-5D show the effect of miR-29a and miR-29c on the level of Mcl-1 proteins. H9c2 cells were transfected with miR-29 mimicking or inhibitory oligonucleotides for 24 h. Results were representative of four independent experiments. FIGS. 5A-5B are samples of immunoblots and FIGS. 5C-5D are densitometric measurements of western blots of Mcl-1 expression. * p<0.05 versus control.

FIG. 6 shows the effect of antagomir-29a (anta-29a), antagomir-29c (anta-29c) and mis-sense antagomir (misanta-29) on myocardial infarct size (% of the ischemic area at risk). Agents were administered once daily for 3 days. On the fourth day mice underwent 30 min coronary artery occlusion and 4 h reperfusion. Area at risk was assessed by blue dye. Infarct size was assessed by TTC. There were significant differences among groups (p<0.001). * p<0.05 versus the control group (n=6 in each group).

FIG. 7 shows the effects of antagomir 29 injection during coronary occlusion on myocardial infarct size (% of the ischemic area at risk).

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, subject” means a human or non-human animal selected for treatment or therapy.

As used herein, “subject suspected of having” means a subject exhibiting one or more clinical indicators of a disease or condition.

As used herein, “preventing” or “prevention” refers to delaying or forestalling the onset, development or progression of a condition or disease for a period of time, including weeks, months, or years.

As used herein, “treatment” or “treat” means the application of one or more specific procedures used for the cure or amelioration of a disease. In certain embodiments, the specific procedure is the administration of one or more pharmaceutical agents.

As used herein, “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

As used herein, “subject in need thereof” means a subject identified as in need of a therapy or treatment.

As used herein, “administering” means providing a pharmaceutical agent or composition to a subject, and includes, but is not limited to, administering by a medical professional and self-administering.

As used herein, “parenteral administration,” means administration through injection or infusion.

As used herein, “parenteral administration includes, but is not limited to, subcutaneous administration, intravenous administration, intramuscular administration, intraarterial administration, and intracranial administration.

As used herein, “subcutaneous administration” means administration just below the skin.

As used herein, “intravenous administration” means administration into a vein.

As used herein, “pharmaceutical composition” means a mixture of substances suitable for administering to an individual that includes a pharmaceutical agent.

As used herein, “pharmaceutical agent” means a substance that provides a therapeutic effect when administered to a subject.

As used herein, “miRNA” or “miR” means a non-coding RNA between 18 and 25 nucleobases in length which hybridizes to and regulates the expression of a coding RNA. In certain embodiments, a miRNA is the product of cleavage of a pre-miRNA by the enzyme Dicer. Examples of miRNAs are found in the miRNA database known as miRBase (http://microrna.sangerac.uk/).

As used herein, “pre-miRNA” or “pre-miR” means a non-coding RNA having a hairpin structure, which contains a miRNA. In certain embodiments, a pre-miRNA is the product of cleavage of a pri-miR by the double-stranded RNA-specific ribonuclease known as Drosha.

As used herein, “antisense compound” means a compound having a nucleobase sequence that will allow hybridization to a target nucleic acid. In certain embodiments, an antisense compound is an oligonucleotide having a nucleobase sequence complementary to a target nucleic acid.

As used herein, “oligonucleotide” means a polymer of linked nucleosides, each of which can be modified or unmodified, independent from one another.

As used herein, “miR antagonist” means an agent designed to interfere with or inhibit the activity of a miRNA. In certain embodiments, a miR antagonist comprises an antisense compound targeted to a miRNA. In certain embodiments, a miR antagonist comprises a modified oligonucleotide having a nucleobase sequence that is complementary to the nucleobase sequence of a miRNA, or a precursor thereof. In certain embodiments, a miR antagonist is a miR-29 antagonist. In other embodiments, an miR-29 antagonist comprises a small molecule, or the like that interferes with or inhibits the activity of an miRNA.

Accordingly, the present invention is directed to a method of treating a vascular disease or injury in an individual in need of such treatment, comprising the step of administering a compound or composition that down-regulates or decreases the activity of microRNA-29 in said individual.

In one aspect, the microRNA-29 is microRNA-29a. In another aspect, the microRNA-29 is microRNA-29c. In one embodiment, the vascular disease or injury is a cardiovascular disease or injury such as but not limited to a myocardial infarction or ischemia reperfusion injury. In another embodiment, the vascular disease or injury is a cerebrovascular disease or injury such as but not limited to stroke. In one embodiment, the compound or composition that down-regulates or decreases the activity of microRNA-29 is a PPAR-γ agonist. Representative PPAR-γ agonists include but are not limited to pioglitazone, 15-deoxy-delta-12,14-PGJ₂ or a thiazolidinedione. In another embodiment, the compound or composition that down-regulates or decreases the activity of microRNA-29 is a microRNA-29 antagonist. Although the microRNA-29 antagonist may be adminstered by any acceptable means, a preferred means of administration is intravenous. The microRNA-29 antagonist may be a miR-29a antisense oligonucleotide or a miR-29c antisense oligonucleotide. Down-regulating or decreasing the activity of microRNA-29 results in up-regulation of Mcl-1 and attenuation of caspase-3 activation in said individual.

The present invention is also directed to a method of down-regulating or decreasing the activity of microRNA-29 in an individual in need of such treatment, comprising the step of administering a PPAR-γ agonist compound or PPAR-γ agonist containing composition that down-regulates or decreases the activity of microRNA-29 in said individual. In one aspect, the microRNA-29 is microRNA-29a. In another aspect, the microRNA-29 is microRNA-29c. In one embodiment, the vascular disease or injury is a cardiovascular disease or injury such as but not limited to a myocardial infarction or ischemia reperfusion injury. In another embodiment, the vascular disease or injury is a cerebrovascular disease or injury such as but not limited to stroke. Representative PPAR-γ agonists include pioglitazone, 15-deoxy-delta-12,14-PGJ₂ or a thiazolidinedione. In another embodiment, the compound or composition that down-regulates or decreases the activity of microRNA-29 is a microRNA-29 antagonist. Although the microRNA-29 antagonist may be adminstered by any acceptable means, a preferred means of administration is intraveneous. The microRNA-29 antagonist may be a miR-29a antisense oligonucleotide or a miR-29c antisense oligonucleotide. Down-regulating or decreasing the activity of microRNA-29 results in up-regulation of Mcl-1 and attenuation of caspase-3 activation in said individual.

The present invention is further directed to a method of treating a vascular disease or injury in an individual in need of such treatment, comprising the step of administering a microRNA-29 antagonist that down-regulates or decreases the activity of microRNA-29 in said individual. In one aspect, the microRNA-29 is microRNA-29a. In another aspect, the microRNA-29 is microRNA-29c. In one embodiment, the vascular disease or injury is a cardiovascular disease or injury such as but not limited to a myocardial infarction or ischemia reperfusion injury. In another embodiment, the vascular disease or injury is a cerebrovascular disease or injury such as but not limited to stroke. In one embodiment, the compound or composition that down-regulates or decreases the activity of microRNA-29 is a PPAR-γ agonist. Representative PPAR-γ agonists include pioglitazone, 15-deoxy-delta-12,14-PGJ₂ or a thiazolidinedione. Although the microRNA-29 antagonist may be adminstered by any acceptable means, a preferred means of administration is intravenous. The microRNA-29 antagonist may be a miR-29a antisense oligonucleotide or a miR-29c antisense oligonucleotide. Down-regulating or decreasing the activity of microRNA-29 results in up-regulation of Mcl-1 and attenuation of caspase-3 activation in said individual.

The following examples are given for the purpose of illustrating various embodiments of the invention and are not meant to limit the present invention in any fashion.

### Example 1

### Animal Care

All animals received humane care in compliance with The Guide for the Care and Use of Laboratory Animals, published by National Institutes of Health (NIH Publication No. 85-23, Revised 1996). Experiments were conducted on male Sprague-Dawley rats.

### Example 2

### Materials

Dharma FECT transfection reagent, miRNA inhibitor, miRNA mimics and non-targeting controls, anatagomir-29a, antagomir-29c and mis-sense antagomir were purchased from Dharmacon (Dharmacon, Inc Ill.) (14-15). Northern kit was purchased from Signosis (Sunnyvale, Calif.). QRT-PCR kit was purchased from Cayman Chemical (Ambion, Austin, Tex.). Monoclonal anti-Mcl 1 antibody was purchased from Santa Cruz Biotechnology (Santa Cruz, Calif.); Pioglitazone (PIO) was purchased from Takeda (Lincolnshire, Ill.). Monoclonal anti-β-actin antibody, GW9662 and protease inhibitor cocktail were purchased from Sigma (St. Louis, Mo.). H9c2 cells and complete growth medium were purchased from American Type Culture Collection (ATCC, Manassas, Va.). Trizol Reagent was purchased from Invitrogen (Carlsbad, Calif.). MTT cell respiration assay kit was purchased from R&D system (Minneapolis, Minn.), dUTP nick end-labeling (TUNEL) assay kit from Roche (Indianapolis, Ind.), and Caspase-3 Colorimetric Protease Assay Kit from BioSource (Hopkinton, Mass.).

### Example 3

### MiRNA Microarray

Rats received 7-day pretreatments with 1) PIO 5 mg·kg⁻¹·day⁻¹; or 2) water (control). Medications were suspended in 1 ml of water and given once a day by oral gavage. There were 3 rats in each group. Total RNA including small RNAs were extracted from left ventricle by using Trizol Reagent. 10 μg of RNA samples were sent to L.C. Sciences for microRNA microarray (10). Samples were enriched for small RNA. Each pair of samples (Control and PIO) were labeled with Cys3 and Cys5 fluorescent dyes and hybridized to a single Atactic μParaFlo microfluidics chip that held all 334 mature rodent miRNA probes. Perfectly matched and mismatched probes were used for quality control. The raw data represented the average of 9 signal values for each miRNA on the array. The background was subtracted and normalized using LOWESS (locally weighted regression) method. The t test was used to compare between control and PIO groups, t values were calculated for each miRNA.

### Example 4

### Northern Blotting Analysis

Rats received pretreatment with 1) PIO 5 mg·kg⁻¹·day⁻¹ for 3 or 7 days; or 2) water (control) for 7 days. Total RNA were isolated using the Trizol protocol (Invitrogene) and northern blot analysis were performed using Northern blot Blot Assay Kit (Signosis Inc, Sunnyvale, Calif.) according to the manufacturer's instructions. Biotin pre-labeled probes for miR-29 and loading control U6 RNA were purchased from Signosis. Membranes were exposed using a chemiluminescence imaging system (Ultralum, Inc. Claremont, Calif.). Expression levels were evaluated using the Typhoon Phosphor Imager software analysis (Amersham Biosciences, Piscataway, N.J.).

### Example 5

### Determination of MiR-29 Expression Level in H9c2 Cells

The cardiac muscle cell H9c2 was maintained in complete growth medium, supplemented with 10% FBS, penicillin G (100 IU/ml). Cells were incubated at 37° C. in a humidified atmosphere (5% CO₂/95% air). Passages 5-9 were used for all experiments. Exponentially growing H9c2 cells were seeded at 1×10⁵ per 100 mm plate and incubated until 60-70% confluent. Cells were treated with: 1) vehicle alone (2% ethanol); 2) 10 nM PIO; 3) 1 μM GW9662; 4) 10 nM PIO+1 μM GW9662. PIO was dissolved in 70% ethanol. 24 hours later, cells were harvested and total RNA enriched with small RNAs was isolated using mirVana miRNA Isolation Kit (Ambion). MiR-29 levels were measured using the mirVana qRT-PCR miRNA Detection Kit (Ambion). Reactions containing mirVana qRT-PCR Primer Sets were specific for rat miR-29. The appropriate cycle threshold (Ct) was determined by using the automatic baseline determination feature. The expression of miR-29 between RNA samples was calculated. Data were normalized by evaluating U6 expression.

### Example 6

### Transfection and Treatment Groups

Exponentially growing H9c2 cells were seeded at 1×10⁶ per 100 mm plate or at 1×10⁵ per 96-wells plate overnight until 50% confluent. Then, the cells were exposed to various agents for the transfection experiments. 10 nM of miR-29 inhibitor, mimic and non-targeting controls were placed in tube 1 and the transfection reagent placed in tube 2 with serum-free medium. The contents in tubes 1 and 2 were combined and incubated for 20 min at room temperature. After removing the growth medium, complete medium together with the mixture from the above were then added to the plate. The cells were incubated at 37° C. with 5% CO2 for 24 h with antibiotic-free medium.

Protocol 1: 1) negative control for mimic (NC-mim); 2) negative control for inhibitor (NC-inh); 3) miR-29a mimic (mim-29a); 4) miR-29a inhibitor (inh-29a); 5) miR-29c mimic (mim-29c); 6) miR-29c inhibitor (inh-29c). Protocol 2: 1) vehicle alone (2% ethanol); 3) PIO 10 nM; 4) PIO+mim-29a; 5) PIO+mim-29c; 6) PIO+mim-29a+mim-29c.

### Example 7

### Simulating Ischemia-Reperfusion (SIR)

Twenty four hours after transfection, cells were subjected to simulated ischemia-reperfusion (SIR): 16 h hypoxia and 2 h reoxygenation or 18 h incubation in normoxemic condition (NSIR). Hypoxia consisted of layering mineral oil over a thin film of hypoxic media (pre-bubbled with N2 gas) that covered the cells for 16 h. The hypoxic media was a HEPES-buffered medium that contained (in mmol/L) 139 NaCl, 4.7 KCl, 0.5 MgCl2, 0.9 CaCl2, 5 HEPES, pH 7.4. The hypoxia medium was replaced by fresh medium (complete growth medium) and followed by 2 h reoxygenation. The hypoxia/reoxygenation-induced cell death was measured by counting trypan blue stained cells and expressed as a percentage of the total cells counted, based on the ability of live cells to exclude trypan blue.

### Example 8

### Determination of Cell Viability

Cell viability was measured by MTT cell viability assay. The cells were treated with 3-[4,5-yl]-2,5-diphenyltetrazolium bromide (MTT, 0.5 mg/ml) for 4 h at 37° C. The attached cells were lysed in 2-isopropanol containing 0.04 M HCl and the amount of metabolized MTT was determined using a micro-plate reader.

### Example 9

### Apoptotic Cell Detection by in Situ End-Labeling and Nuclear Staining

To detect programmed cell death, the apoptotic nuclei were labeled using terminal deoxy-nucleotidyl transferase-mediated dUTP nick end-labeling (TUNEL) assay according to the manufacturer's instructions. The nuclei of apoptotic and non-apoptotic cells were counterstained with DAPI (0.1 μg/ml). The labeled cells were counted under a fluorescence microscope. The percentage of apoptotic cells was calculated as the ratio of TUNEL-positive cells to the DAPI-stained total cells, counted in six different random fields.

### Example 10

### Measurement of Caspase-3 Activity

The enzymatic activity of caspase-3 induced by various conditions was measured with the Caspase-3 Colorimetric Protease Assay Kit according to the manufacturer's instructions. H9c2 cells prepared after respective treatments were lysed in a lysis buffer (1% Triton X-100, 0.32 M sucrose, 5 mM EDTA, 1 mM phenylmethylsulfonyl fluoride, 1 μM aprotinin, 1 μM leupeptin, 2 mM dithiothreitol, 10 mM Tris/HCl, pH 8.0) on ice for 30 min and centrifuged at 15,000×g for 15 min. To determine the activity of caspase-3, assays were performed by incubating 200 μg protein of cell lysate in 100 μl of reaction buffer containing 5 μl of caspase-3 substrate (4 mM DEVD-pNA) in 96-well plates. The reaction buffer contained 1% NP-40, 20 mM Tris-HCl (pH 7.5), 137 mM N-acetyl-cysteine and 10% glycerol. Lysates were incubated at 37° C. for 2 h. The samples were incubated in the dark and measured with a micro-plate reader at an absorbance of 405 nm.

### Example 11

### Western Blot Analysis

Mcl-1 protein expression was determined by immunobloting. Samples were homogenized in lysis buffer (in mMol): 25 Tris.HCl (pH 7.4), 0.5 EDTA, 0.5 EGTA, 1 phenylmethylsulfonyl fluoride, 1 dithiothreitol, 25 NaF, 1 Na₃VO₄, 1% Triton X-100, 2% SDS and 1% protease inhibitor cocktail. The lysate was centrifuged at 10,000 g for 15 min at 4° C. The resulting supernatants were collected. Protein (75 μg) was fractionated by SDS-PAGE (4%-20% polyacrylamide gels) and transferred to PVDF membranes (Millipore, Bedford, Mass.). The samples were incubated overnight at 4° C. with Anti-Mcl 1 antibodies. Bound antibodies were detected using the chemiluminescent substrate (NEN Life Science Products, Boston, Mass.). The protein signals were quantified with an image-scanning densitometer, and the strength of each protein signal was normalized to the corresponding _-actin signal. Data are expressed as percent of the expression in the control group.

### Example 12

### Infarct Size

Mice received intravenous injection of 80 mg/kg of antagomir-29a, antagomir-29c, mis-sense antagomir, or vehicle alone (PBS) once daily for 3 days. Last injection was given 16 h before surgery. Similar regimen has been previously shown to be effective, and to effectively silence microRNAs up to 24 h after intravenous injection (14-15). On the fourth day mice were anesthetized with intraperitoneal injection of ketamine (60 mg/kg) and xylazine (6 mg/kg), intubated and ventilated (FIO₂=30%). The rectal temperature was monitored and body temperature was maintained between 36.7 and 37.3° C. throughout the experiment. The chest was opened and the left coronary artery was encircled with a suture and ligated for 30 minutes. Ischemia was verified by regional dysfunction and discoloration of the ischemic zone. Isofluorane (1-2.0% titrated to effect) was added after the beginning of ischemia to maintain anesthesia. At 30 minutes of ischemia, the snare was released and myocardial reperfusion was verified by change in the color of the myocardium. Subcutaneous 0.1 mg/kg buprenorphine was administered, the chest was closed and the mice were recovered from anesthesia. Four hours after reperfusion the mice were re-anesthetized, the coronary artery was reoccluded, Evan's blue dye 3% was injected into the right ventricle and the mice euthanized under deep anesthesia (13,17-18). The pre-specified exclusion criteria were lack of signs of ischemia during coronary artery ligation, lack of signs of reperfusion after release of the snare, prolonged ventricular arrhythmia with hypotension, and area at risk ≦ 10% of the LV weight.

### Example 13

### Determination of Area at Risk (AR) and Infarct Size (IS)

Hearts were excised and the left ventricle was sliced transversely into 6 sections. Slices were incubated for 10 minutes at 37° C. in 1% buffered (pH=7.4) 2,3,5-triphenyl-tetrazolium-chloride (TTC), fixed in a 10% formaldehyde and photographed in order to identify the area at risk (uncolored by the blue dye), the infarct size (unstained by TTC), and the non-ischemic zones (colored by blue dye). The area at risk and infarct size in each slice were determined by planimetry, converted into percentages of the whole for each slice, and multiplied by the weight of the slice and the results summed to obtain the weight of the myocardial area at risk and infarct size (13, 17-18).

### Example 14

### Statistical Analysis

Data are presented as means ± standard errors of means. Analysis of variance (ANOVA) with Sidak correction for multiple comparisons was applied to compare the different groups. P<0.05 was considered statistically significant.

### Example 15

### MiR-29 Expressions is Down-Regulated by PIO

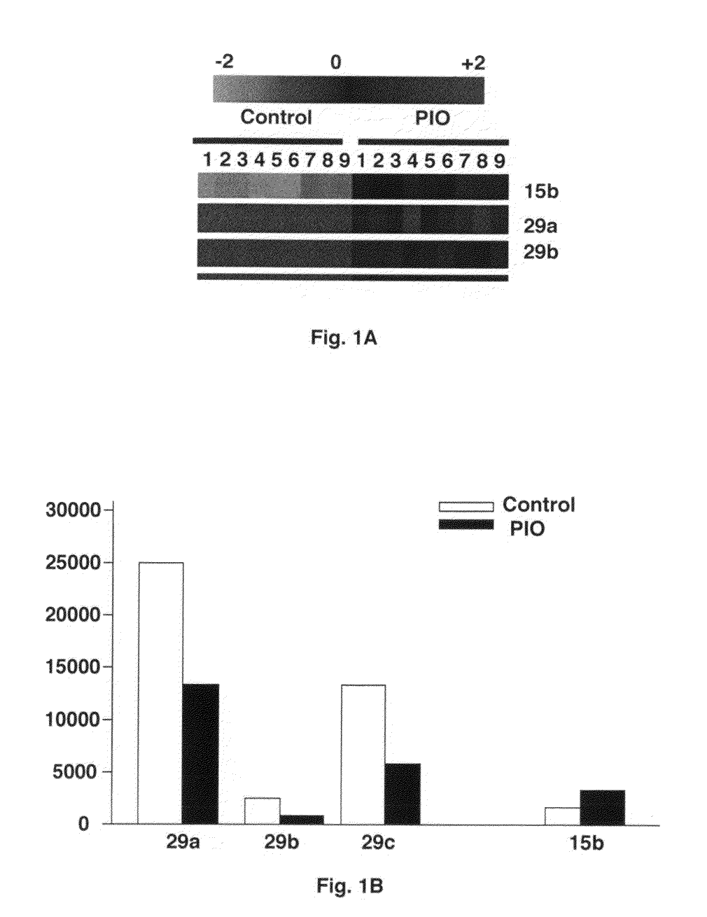

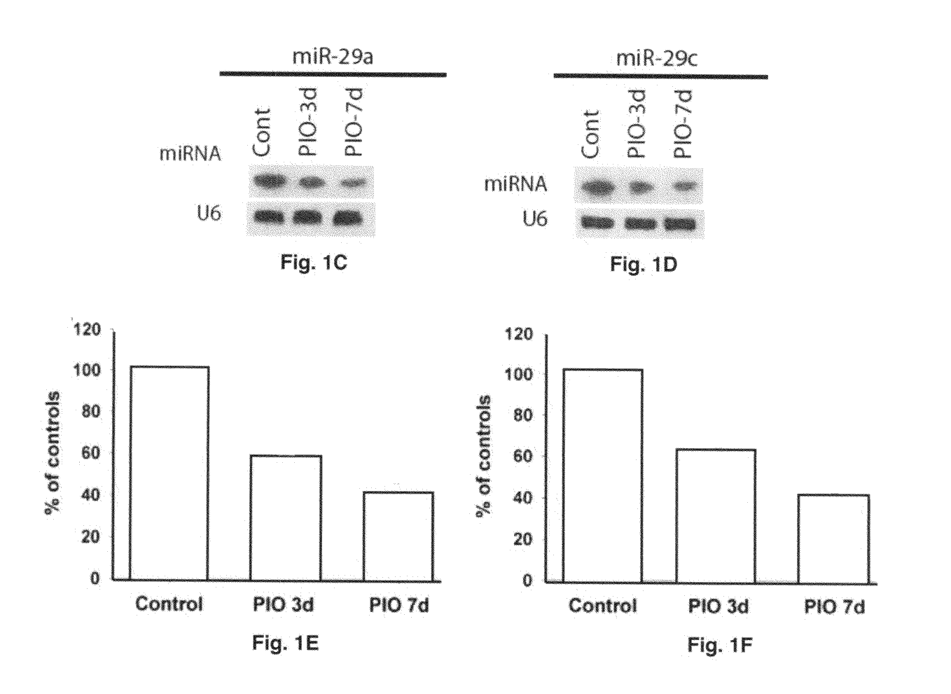

A miR-29 family was first identified as a candidate miRNA down-regulated by PIO by using miRNA arrays screening. As showed in (FIGS. 1A-1B), pretreatment with PIO caused a significant reduction in the myocardial expression of miR-29a, -29b, -29c and augmentation of miR-15b compared with control. Based on their expression levels relative to the PIO-treated values, miR-29a & -29c was examined. Northern blot analyses of cardiac RNA from rat left ventricle confirmed decreases in the expression of miR-29a and miR-29c after PIO administration (FIGS. 1C-1D).

### Example 16

### Down-Regulation Effect of PIO on miR-29 Expression Blocked by the PPAR-Inhibitor GW9662

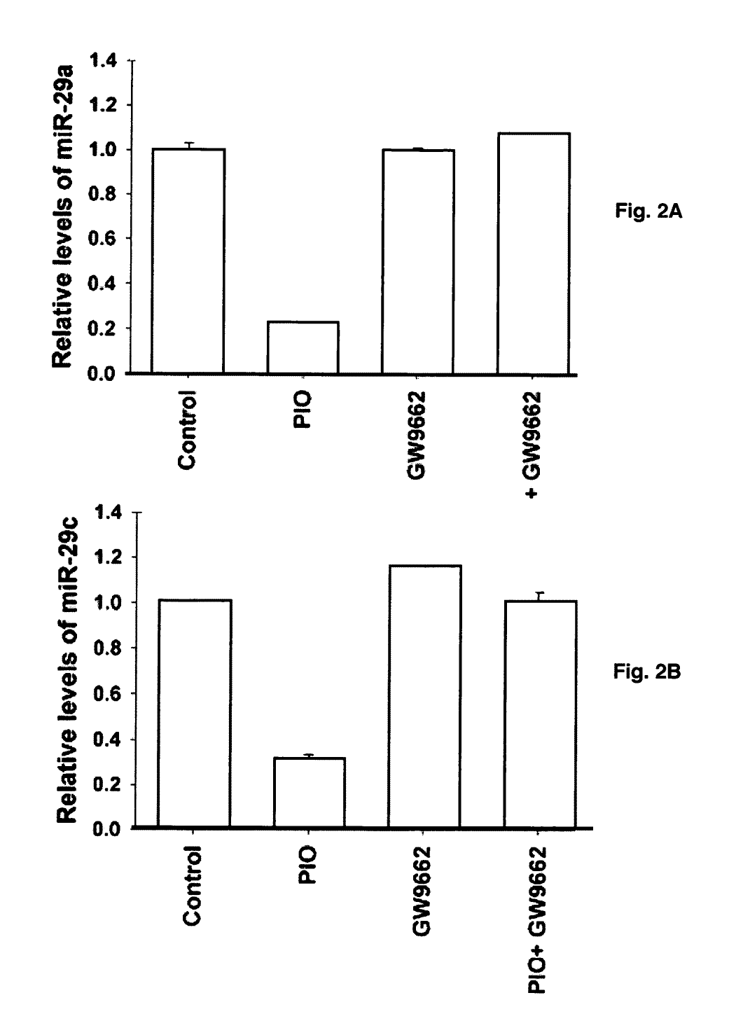

The effect of GW9662, a selective PPAR-γinhibitor, was also assessed. As indicated in FIGS. 2A-2B, QRT-PCR revealed that miR-29a and miR-29c were highly expressed in H9c2 cells and remarkably decreased by the PIO treatment. GW9662 alone had no effect; however, it did block the effect of PIO on miR-29 levels.

### Example 17

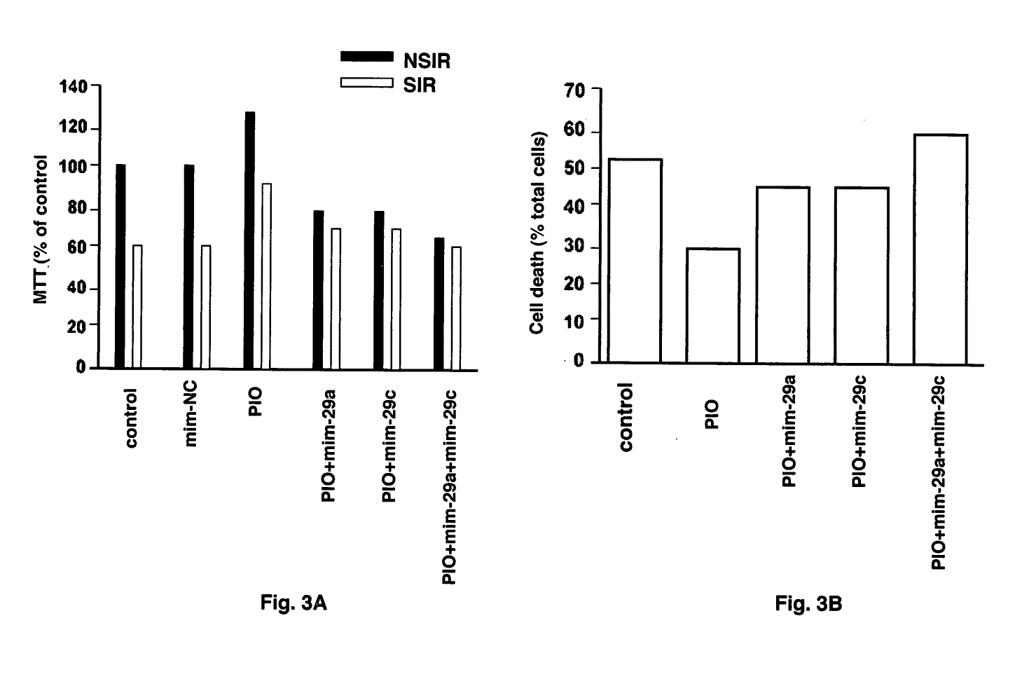

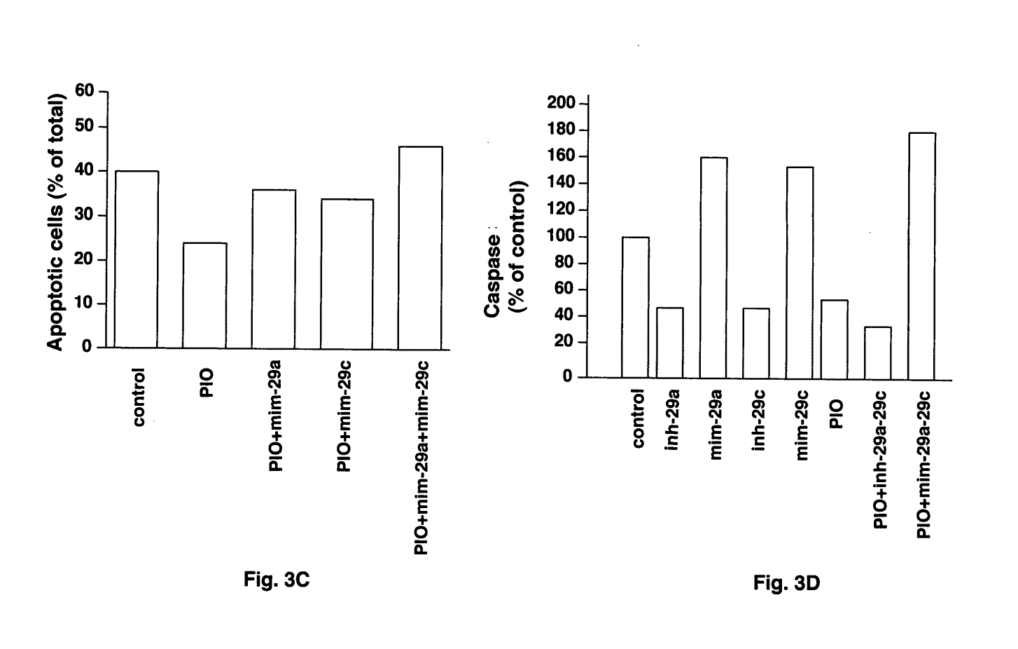

### Over-Expression of miR-29a and miR-29c Blocks the Protective Effect of PIO Against SIR PIO strongly improved the viability of cells subjected to SIR (FIG. 3A). Mim-29a or mim-29c partially blocked the protective effect of PIO. The combination of mim-29a and -29c abolished the protective effect of PIO on SIR. PIO significantly reduced death of cells exposed to SIR (FIG. 3B). The protective effect of PIO was partially blocked by mim-29a and mim-29c and completely blocked by co-transfection with mim-29a and mim-29c.

Apoptotic cell death was verified by TUNEL assay for chromosomal cleavage (FIG. 3C). PIO significantly decreased the number of positively stained cells after exposure to SIR. Transfection with mim-29a or mim-29c respectively, partially blocked the effect of PIO. Whereas, co-transfection with mim-29a and mim-29c completely blocked the anti-apoptotic effect of PIO.

Caspase-3 activity was analyzed in all groups subjected to SIR. Compared to the control group, inhibition of miR-29 by inh-29a or inh-29c caused a significant decrease in caspase-3 activity (FIG. 3D). PIO also significantly reduced caspase activity. The effect of PIO on caspase-3 activity was blocked by transfection with the combination of mim-29a and miR-29c; whereas it was augmented by combining PIO with inh-29a and inh-29c.

### Example 18

### Down-Regulation of miR-29 by Antisense Inhibitor Promotes Cell Viability and Prevents Cell Death Induced by SIR

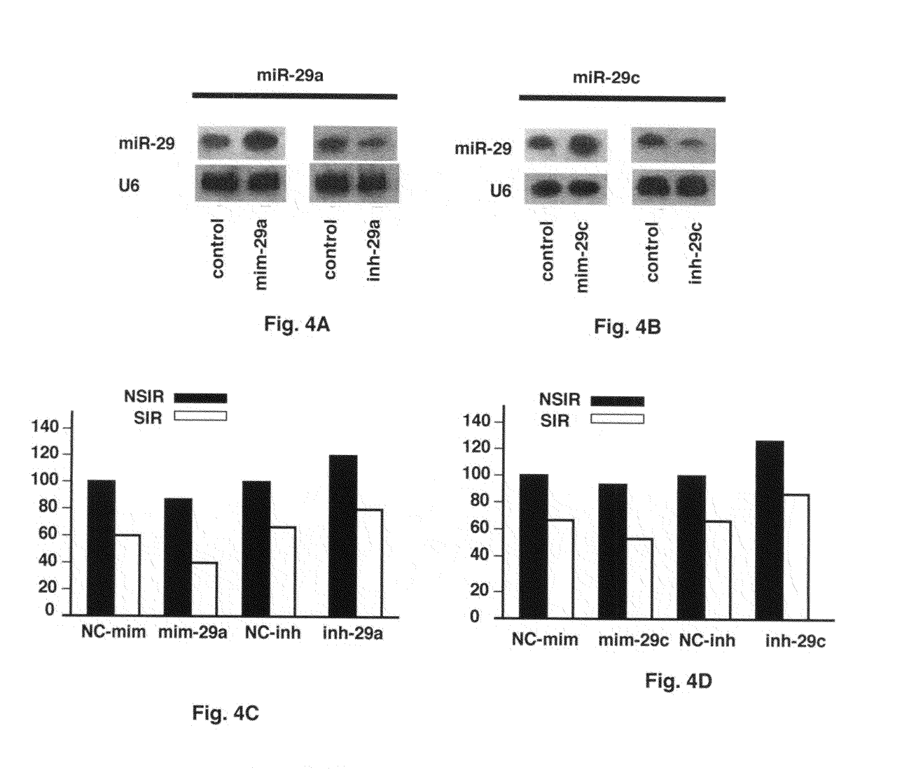

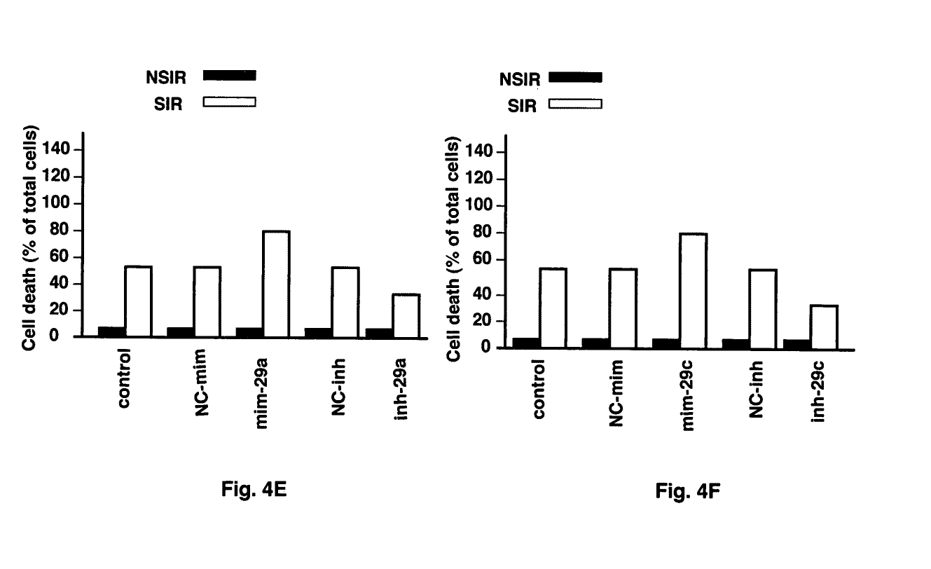

To assess the functional consequences of silencing or overexpression of miR-29 in SIR, antisense inhibitor and mimic oligonucleotides were transfected into H9c2 cells. Northern blot assays confirmed that miR-29a, or miR-29c, expression levels were increased 3 fold by mim-29a and mim-29c when compared with negative control mimic (NC-mim). In contrast, miR-29a and miR-29c levels were decreased by the specific inh-29a and inh-29c, respectively (FIGS. 4A-4B). After transfection, cells were subjected to SIR or NSIR. As indicated in FIGS. 4C-4D, SIR reduced cell viability. Mim-29a and mim-29c treatment also reduced cell viability in both SIR and NSIR groups. On the other hand, inh-29a and inh-29c treatment increased cell viability in both the SIR and NSIR groups. Negative control had no effect. SIR induced cell death, as assessed by trypan blue staining (FIGS. 4E-4F). Mim-29 or inh-29 did not affect total cell death in the NSIR groups. However, mim-29a and mim-29c augmented cell death induced by SIR. In contrast, inh-29a and inh-29c significantly reduced cell death in cells exposed to SIR.

### Example 19

### MiR-29 Downregulates Mcl-1 Protein

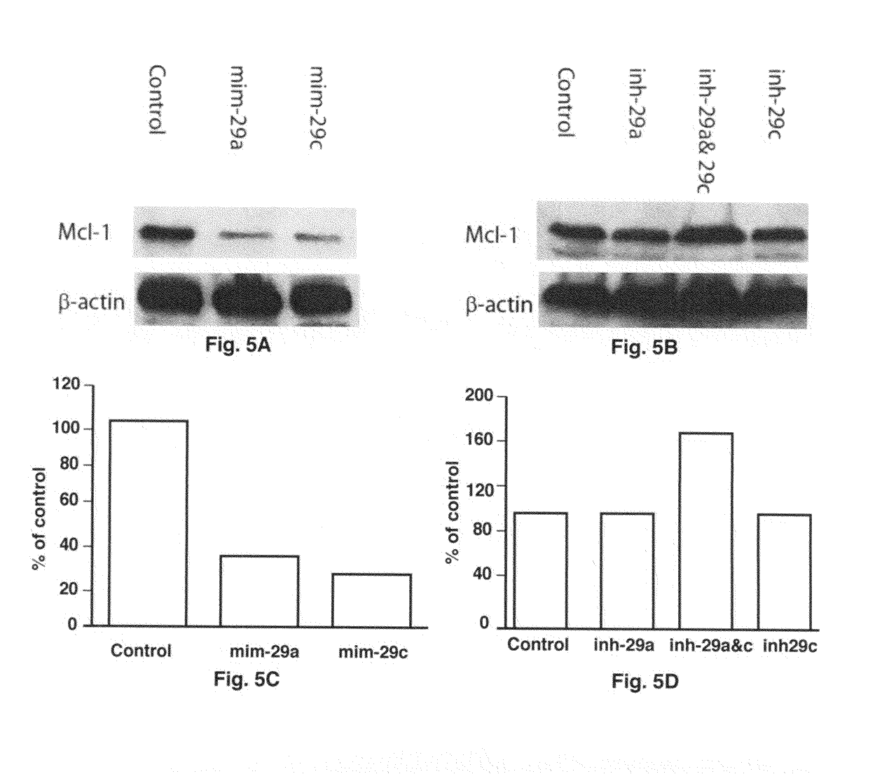

Transfection with the miR-29a or miR-29c produced a remarkable decrease in Mcl-1 protein levels (FIGS. 5A and 5C). Transfection with either inh-29a or inh-29c did not cause significant changes in Mcl-1 protein levels (FIGS. 5B and 5D). Co-transfection with inh-29a & inh-c, however, was able to increase the level by 67% (p<0.001).

### Example 20

### The Effect of Pretreatment with Antagomir-29a and Antagomir-29c on Infarct Size

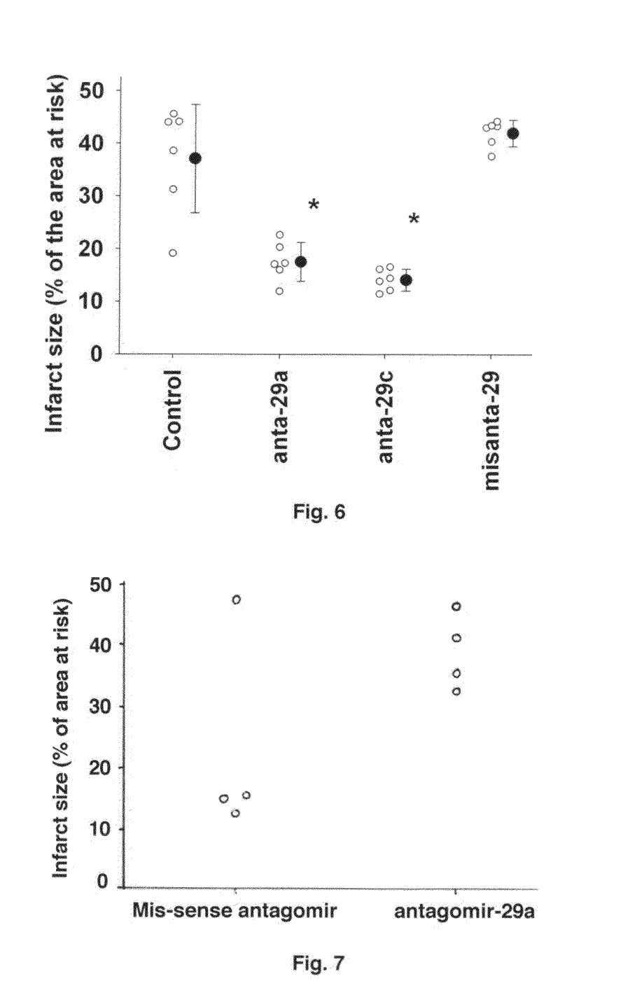

A total of 24 mice were included (6 in each group). None of the mice died or were excluded. Body weight and the size of the area at risk were comparable among groups. IS was significantly smaller in the antagomir-29a and antagomir-29c treated group than in the control group (receiving PBS injection), whereas mis-sense antagomir had no effect (FIG. 6).

### Example 21

### The Effect of Antagomir-29a and Antagomir-29c on Infarct Size

A total of 8 mice were included (4 in each group). Mice underwent 30 minutes of coronary artery occlusion and 4 hours of reperfusion. At 5 minutes of occlusion mice received intravenous antagomir-29a or mis-sense antagomir (FIG. 7). Infarct size (% of the ischemic area at risk) was significantly smaller in the antagomir-29a (p<0.0001).

### Discussion

Pharmacological agents targeting pro-apoptotic pathways in IRI can exert a cardioprotective effect. Instead of using ischemic preconditioning, which is a robust stimulation that activates numerous pathways (some which are related to the injury, whereas other mediate protection), an established model of pharmacological preconditioning with PIO was used. Thiazolidinedione such as pioglitazone limit myocardial infarct size (9-10, 13, 17, 19), suggesting a protective role of thiazolidinediones against IRI. However, the underlying mechanisms of this protective effect are not fully understood. The present study demonstrates for the first time that miR-29a and miR-29c are involved in IR1.

At first it was shown that PIO decreased miR-29a and miR-29c levels in the rat heart. MiRNA gene microarray showed that treatment with PIO increased levels of miR-15b and decreased the levels of miR-29a, miR-29b, and miR-29c in rat hearts. The results were confirmed by northern blot analysis. As levels of miR15b and miR-29b in the rat heart were much lower than levels of miR-29a and miR-29c, the role of miR-29a and miR-29c was examined.

Subsequently, using an in-vitro model of SIR injury, the PIO-induced down-regulation of miR-29 expression was blocked by the PPARγ inhibitor GW9662, suggesting that it is PPARγ dependent. Transfecting H9c2 cells with miR-29a and miR-29c mimic oligonucleotides induced apoptosis and cell death and exacerbated IRI, whereas transfection with miR-29a and miR-29c antisense oligonucleotides reduced apoptosis and cell death and conferred resistance to IRI. Moreover, the protective effect of PIO against SIR injury was blocked by overexpressing miR-29 mimic oligonucleotides, suggesting an essential role for the inhibition of miR-29 in the cardioprotective effect of PIO against IRI. Thus, data showing the involvement of miRNA in IR1 is provided as well as a novel pharmacological approach to modulate miRNA levels using a PPAR-γ agonist. Finally, an in vivo model was used to show that intravenous injection of antagomir-29a or 29c limits myocardial infarct size when given for 3 days before myocardial ischemia and even when administered intravenously after ischemia has started, before reperfusion.

As some of the “pleiotropic” effects of PIO are PPAR-γ independent²⁰⁻²², whether the effect of PIO on miR-29s expression is PPAR-γ dependent was examined. GW9662, a specific PPAR-γ antagonist, attenuated the effect of PIO; thus, it seems that the PIO effect on miR-29 expression is PPAR-γ dependent. GW9662 does not block the augmentation of prostaglandin D₂ and 15-deoxy-delta-12,14-PGJ₂ levels by PIO, suggesting that this effect is independent of PPAR-γ activation (23). 15-deoxy-delta-12,14-PGJ₂ is the natural ligand of PPAR-γ and has numerous PPAR-γ dependent and independent beneficial effects including protection against IRI (10). It is anticipated that 15-deoxy-delta-12,14-PGJ₂ and other PPAR-γ agonists, including other thiazolidinediones, have the same effects on miR-29 levels.

Increasing evidence suggests that miRNAs play pivotal roles in various heart diseases by altering transcription of important genes (2, 7, 24). To extend these concepts, these data demonstrated that down-regulation of miR-29 by PIO or by antisense inhibitor helps H9c2 myocyte evade cell death following SIR injury. To study the biological significance of miR-29 expression, a mimic or inhibitor of miR-29 to over-expressed or silence miR-29 was employed. Transfection with miR-29a and miR-29c mimic induced apoptosis and cell death and exacerbated IRI, whereas transfection with miR-29a and miR-29c antisense inhibitors reduced apoptosis and cell death and conferred resistance to IRI. Moreover, the protective effect of PIO against SIR injury was blocked by overexpressing miR-29 mimic, suggesting an essential role for the inhibition of miR-29 in the cardioprotective effect of PIO against IRI. Using an in vivo model, antagomir-29a and 29c protected the heart and reduce infarct size in the mouse.

MiR-29b2 and miR-29c are located on chromosome 13 and miR-29b1 and miR-29a are located on chromosome 4 in rats (25). Increased levels of miR-29a, miR-29b, and miR-29c were found in skeletal muscle, fat and liver of Goto-Kakizaki rats with type-2 diabetes mellitus (25). Overexpression of miR-29s in 3T3-L1 adipocytes repress insulin-stimulated glucose uptake (25). Thus, it seems that miR-29s also have a role in mediating insulin resistance. Suppression of miR-29s levels may partially explain the effects of PIO on insulin resistance. Upon insulin treatment of adipocytes overexpressing miR-29, Akt phosphorylation at Ser 473 was attenuated by 59% (25). On the other hand, antisense inhibition of miR-29 in 3T3-L1 cells improved Akt activation in the presence of insulin²⁵. It has recently been shown that miR-29 suppresses the p85α regulatory subunit of phosphoinositol 3 kinase (PI3K) (28).

On the other hand, it has been reported that miR-29 is downregulated in the infarct border zone of hearts explanted from patients undergoing heart transplantation and in mice with experimental coronary ligation (29). Van Rooij suggested that miR-29s are associated with fibrosis-related mRNAs, including genes encoding multiple collagens, fibrillins, and elastin ²⁹. Downregulation of miR-29 induced collagen expression both in vitro and in vivo, whereas overexpression of miR-29 mimic oligonucleotides reduces collagen transcription²⁹. Similar findings have been reported with Akt activation: short-term activation may confer protection against IRI, whereas long-term activation leads to fibrosis and hypertrophy (30-32). However, several studies have suggested that PIO attenuates cardiac fibrosis in animals with hypertension, or after stimulation with angiotensin II (33-35). As PIO is affecting many other genes in addition to miR-29, the net effect is probably different than pure long-term repression of miR-29 expression using genetic approach.

Of particular importance is the suppressive effect of miR-29s on Mcl-1 levels (36). Mcl-1, an anti-apoptotic Bcl-2 family member, is a putative target of miR-29. Mir-29b overexpression reduces Mcl-1 cellular protein levels and sensitizes cancer cells to TRAIL (tumor necrosis factor-related apoptosis-inducing ligand) cytotoxicity. Transfection of non-malignant cells (that express high levels of miR-29) with a miR-29b antisense oligonucleotides increases Mcl-1 levels³⁶. Mcl-1 is involved in late ischemic preconditioning of the heart (37) and in preconditioning of polymorphonuclear leukocytes by heat (38). In the present study, a combination of antisense oligonucleotides for both miR-29a and miR-29c increased the levels of Mcl-1, whereas miR-29a or miR-29c mimic oligonucleotides alone reduced Mcl-1 levels. Mcl-1 levels in the combined antisense treatment group were above the control values because antisense inhibitor removed the basal repression produced by endogenous miR-29. Noticeably, to obtain significant effects on the endogenous Mcl-1 protein level, co-transfection of inhibitors against miR-29a and 29c is required and either of two inhibitors alone failed to affect Mcl-1 level. This can be explained if Mcl-1 is simultaneously repressed by miR-29a and 29c; thus, the removal of one miRNA is not sufficient to reverse the suppression effect of other one. When both miRNAs are removed, the repression can be relieved. It should be emphasized that regulation of Mcl-1 expression is a complex process and our study merely indicates that there is a potential effect of miR-29 on Mcl-1 but does not exclude other regulatory factors.

In accordance with Mcl-1 levels, miR-29a and miR-29c mimic oligonucleotides increased caspase-3 activity in cells exposed to SIR and the combination of miR-29a and miR-29c mimic oligonucleotides completely blocked the protective effect of PIO. In contrast, the use of miR-29a and miR-29c antisense inhibitory oligonucleotides attenuated the activation of caspase-3 following SIR. Caspases are the key executioners of apoptosis activated in cardiomyocytes exposed to an in vitro hypoxia condition that mimics aspects of in vivo ischemia (39).

The present study aimed to characterize the role of miRNAs in modulation of myocardial IRI. The present invention demonstrates a role for miR-29a and miR-29c in modulating IRI and mediating the protective effect of PIO via PPARγ activation. Taken together, these findings indicate that down-regulation of miR-29s is a novel approach for protecting the heart against IRI and reducing infarct size. This is the first demonstration that cardioprotection by PIO is associated with a complex genetic program that results in modulation of miR-29 and target proteins involved in prosurvival and anti-apoptotic pathways. The beneficial effect of down-regulation of miR-29 is associated with up-regulation of an anti-apoptotic molecule Mcl-1 and attenuation of caspase-3 activation. This finding reveals an important role for specific miRNAs in the control of IRI-induced cell death and point to miRNA as potential drug targets for the treatment of heart disease. Inhibiting miRNAs by antisense strategies and especially by pharmacological approaches are likely to emerge as alternative and probably safe methods for conferring short- and intermediate-term protection against IRI, as it is emerging for other cardiac conditions (2-7). The safety of long-term interventions should be also studied. This will guide us into a new era of molecular control of heart pathophysiology that is beyond traditional signaling regulation pathways.

The following references were cited herein:
1. Bartel D P. Cell. 2004, 116(2):281-297.
2. van Rooij et al. J Clin Invest. Vol 117. 2007/09/06 ed, 2007:2369-2376.
3. Divakaran V and Mann D L. Circ Res. 2008, 103(10):1072-1083.
4. Sayed et al. Circ Res. 2007, 100(3):416-424.
5. Thum et al. Circulation. 2007, 116(3):258-267.
6. Bauersachs et al. Eur J Clin Invest. 2007, 37(11):829-833.
7. Yang et al. Nat. Med. 2007, 13(4):486-491.
8. Wang et al., Fa Yi Xue Za Zhi. 2007, 23(5):328-331.
9. Ito et al. 2003; 83(12):1715-1721.
10. Wayman et al., Faseb J. 2002, 16(9), 1027-1040.
11. Yue et al. Diabetes. 2005, 54(2):554-562.
12. Yue et al. Circulation. 2001, 104(21):2588-2594.
13. Ye et al. American Journal of Physiology-Heart and Circulatory Physiology. 2006; 291(3):H1158-H1169.
14. Krutzfeldt et al. Nature. 2005, 438(7068):685-689.
15. Thum et al., Nature. 2008, 456(7224):980-984.
16. Zhou et al. Nucleic acids research. 2004, 32(18):5409-5417.
17. Ye et al. American journal of physiology. 2008, 295(6):H2436-2446.
18. Ye et al. American Journal of Physiology-Heart and Circulatory Physiology. 2008, 295(1):H343-H351.
19. Wynne et al. Journal of cardiovascular pharmacology. 2005, 46(6):817-822.
20. Kapadia et al. Front Biosci. 2008, 13:1813-1826.
21. Li et al. Clinical and experimental pharmacology & physiology. 2008, 35(12):1471-1476.
22. Ye et al. Circulation Research. 2007, 101(5):E76-E76.
23. Ye et al. Prostaglandins & Other Lipid Mediators. 2007, 84(1-2):43-53.
24. van Rooij et al. Science. 2007, 316(5824):575-579.
25. He et al. Mol. Endocrinol. 2007, 21(11):2785-2794.
26. Huang et al. Biomedicine & Pharmacotherapy (Biomedecine & Pharmacotherapy). 2008, 62(1):46-52.
27. Taniguchi et al. Diabetes. 2006, 55(8):2371-2378.
28. Park et al. Nature Structural & Molecular Biology. 2009, 16(1):23-29.
29. van Rooij et al. Proc Natl Acad Sci USA. 2008, 105(35):13027-13032
30. Nagoshi et al. J Clin Invest. 2005, 115(8):2128-2138.
31. Shiojima et al. J Clin Invest. 2005, 115(8):2108-2118.
32. O'Neill et al. J Clin Invest. 2005, 115(8):2059-2064.
33. Caglayan et al. Diabetes. 2008, 57(9):2470-2479.
34. Kato et al. Journal of Hypertension. 2008, 26(8):1669-1676.
35. Nakamoto et al. Hypertens Res. 2008, 31(2):353-361.
36. Mott et al. Oncogene. 2007, 26(42):6133-6140.
37. Stein et al., J Mol Cell Cardiol. 2007, 42(6):1075-1085.
38. Selva et al. Immunology and Cell Biology. 2007, 85(3):257-264.
39. Bialik et al. Circ Res. 1999, 85(5):403-414.

What is claimed is:

1. A method of treating a vascular disease or injury in an individual in need of such treatment, comprising the step of:
administering a compound or composition that down-regulates or decreases the activity of microRNA-29 in said individual.

2. The method of claim 1, wherein the microRNA-29 is microRNA-29a or microRNA-29c.

3. The method of claim 1, wherein the vascular disease or injury is a cardiovascular disease or injury, a myocardial infarction, is ischemia reperfusion injury, is a cerebrovascular disease or injury.

4. The method of claim 3, wherein the cerebrovascular disease or injury is stroke.

5. The method of claim 1, wherein the compound is a PPAR-γ agonist.

6. The method of claim 5, wherein the PPAR-γ agonist is pioglitazone, 15-deoxy-delta-12,14-PGJ₂ or a thiazolidinedione.

7. The method of claim 1, wherein the compound is a microRNA-29 antagonist.

8. The method of claim 7, wherein the microRNA-29 antagonist is adminstered intraveneously.

9. The method of claim 7, wherein the microRNA-29 antagonist is a miR-29a antisense oligonucleotide or a miR-29c antisense oligonucleotide.

10. The method of claim 1, wherein the down-regulating or decreasing the activity of microRNA-29 results in up-regulation of Mcl-1 and attenuation of caspase-3 activation in the individual.

11. A method of down-regulating or decreasing the activity of microRNA-29 in an individual in need of such treatment, comprising the step of:
administering a PPAR-γ agonist compound or PPAR-γ agonist containing composition that down-regulates or decreases the activity of microRNA-29 in the individual.

12. The method of claim 11, wherein the microRNA-29 is microRNA-29a or microRNA-29c.

13. The method of claim 11, wherein the individual has a vascular disease or injury.

14. The method of claim 11, wherein the vascular disease or injury is a cardiovascular disease or injury, is myocardial infarction, ischemia reperfusion injury, cerebrovascular disease or injury.

15. The method of claim 11, wherein the PPAR-γ agonist is pioglitazone, 15-deoxy-delta-12,14-PGJ₂ or a thiazolidinedione.

16. A method of treating a vascular disease or injury in an individual in need of such treatment, comprising the step of:
administering a microRNA-29 antagonist that down-regulates or decreases the activity of microRNA-29 in the individual.

17. The method of claim 16, wherein the microRNA-29 is microRNA-29a or microRNA-29c.

18. The method of claim 16, wherein the vascular disease or injury is a cardiovascular disease or injury, myocardial infarction, ischemia reperfusion injury or cerebrovascular disease or injury.

19. The method of claim 16, wherein the microRNA-29 antagonist is adminstered intraveneously.

20. The method of claim 16, wherein the microRNA-29 antagonist is a miR-29a antisense oligonucleotide.

21. The method of claim 20, wherein the microRNA-29 antagonist is miR-29c antisense oligonucleotide.

22. The method of claim 16, wherein said down-regulating or decreasing the activity of microRNA-29 results in up-regulation of Mcl-1 and attenuation of caspase-3 activation in the individual.
